(19) United States
(12) Patent Application Publication
(10) Pub. No.: US 2026/0053783 A1
(43) Pub. Date: Feb. 26, 2026

(54) PIOGLITAZONE AND MEK INHIBITORS FOR TREATING CANCER

(71) Applicant: SHEBA IMPACT LTD., Ramat-Gan (IL)

(72) Inventors: Dana ISHAY-RONEN, Tel-Aviv (IL); Rakefet BEN-YISHAY, Ashkelon (IL); Sheli ARBILI YARHI, Or-Yehuda (IL); Neta BAR-HAI GOCHMAN, Ramat-Gan (IL)

(21) Appl. No.: 19/306,824

(22) Filed: Aug. 21, 2025

Related U.S. Application Data
(60) Provisional application No. 63/685,403, filed on Aug. 21, 2024.

(51) Int. Cl. A61K 31/4439; A61K 31/4523; A61P 35/00

(52) U.S. Cl. CPC A61K 31/4439; A61K 31/4523; A61P 35/00

(57) ABSTRACT

The present invention is directed to a combination therapy involving Pioglitazone and a MEK inhibitor for the treatment of a cancer patient in need of such a therapy.

### FIELD OF THE INVENTION

This invention relates to a combination of Pioglitazone and MEK inhibitors for inducing adipogenesis and cancer cell differentiation.

### REFERENCES

1. Ahmadian et al., Nature Medicine, vol. 19, no. 5, pp. 557-66, (2013)
2. Aiello Kang, J Exp Med, vol. 216, no. 5, pp. 1016-1026 (2019)
3. Banks et al., Nature, vol. 517, no. 7534, pp. 391-5 (2015)
4. Boumahdi and de Sauvage, Nat Rev Drug Discov, doi: 10.1038/s41573-019-0044-1 (2019)
5. Farmer, Cell Metab. vol. 4, no. 4, pp. 263-73, (2006)
6. Flaherty et al., N Engl J Med, vol. 367, no. 18, pp. 1694-703, (2012)
7. Gavert et al., Nat Cancer, vol. 3, no. 2, pp. 219-231 (2022)
8. Gerstberger et al., Cell, vol. 186, no. 8, pp. 1564-1579, (2023)
9. Haerinck et al., Nat Rev Genet, doi: 10.1038/s41576-023-00601-0 (2023)
10. Infante et al., Lancet Oncol, vol. 13, no. 8, pp. 773-81 (2012)
11. Ishay-Ronen et al., Cancer cell, vol. 35, no. 1, pp. 17-32 (2019)
12. Krebs et al., Nat Cell Biol, vol. 19, no. 5, pp. 518-529 (2017)
13. Lito et al., Nature Medicine, vol. 19, no. 11, pp. 1401-9, (2013)
14. Morrison and Farmer, J Biol Chem, vol. 274, no. 24, pp. 17088-97, (1999)
15. Nieto, Science, vol. 342, no. 6159, p. 1234850 (2013)
16. Ribas et al., Lancet Oncol, vol. 15, no. 9, pp. 954-65, (2014)
17. Rosen and MacDougald, Nat Rev Mol Cell Biol, vol. 7, no. 12, pp. 885-96 (2006)

### BACKGROUND OF THE INVENTION

Cancer cells have the remarkable ability to change their phenotype and adapt to external signals from the microenvironment, a phenomenon known as cancer cell plasticity (Haerinck et al., 2023; Gerstberger et al., 2023; Boumahdi and de Sauvage, 2019). Epithelial-to-mesenchymal transition (EMT) and its reversal mesenchymal-to-epithelial transition (MET) are essential processes in embryonic development and can be reactivated and utilized by cancer cells to enhance cellular plasticity and malignant progression (Haerinck et al., 2023; Nieto, 2013). EMT in breast cancer is particularly associated with tumor invasion, metastasis and drug resistance (Aiello and Kang, 2019).

Ishay-Ronen et al (2019) demonstrated a novel approach to overcome cancer cell plasticity by converting invasive breast cancer cells into post-mitotic functional adipocytes through the combined use of Rosiglitazone and Trametinib. The trans-differentiation approach effectively repressed breast cancer progression and metastasis formation in murine models and human subjects.

Adipogenesis is the process by which preadipocytes differentiate into mature adipocytes. This process is regulated by several transcription factors, including CCAAT-enhancer binding protein α (C/EBPα) and PPARγ, which are considered master regulators of adipogenesis (Farmer 2006; Morrison and Farmer 1999). PPARγ activation is essential for adipogenesis, as it initiates the transcriptional cascade that leads to the terminal differentiation of adipocytes.

Pioglitazone is a synthetic PPARγ agonist with potent insulin-sensitizing effects and has been used in the treatment of type 2 diabetes (Ahmadian et al., 2013). Cobimetinib, Binimetinib, Selumetinib, and Trametinib inhibit the MEK-ERK signaling pathway, which is often dysregulated in cancer (Flaherty et al., 2012; Lito et al., 2013; Infante et al., 2012; Ribas et al., 2014).

### SUMMARY OF THE INVENTION

In one aspect, the present invention provides a combination of Pioglitazone or a pharmaceutically acceptable salt thereof and a MEK inhibitor or a pharmaceutically acceptable salt thereof, for use in treating or preventing cancer, said treating or preventing cancer comprises administering to a mammal in need thereof a therapeutically effective amount of said combination.

In another aspect, the present invention provides a method of treating or preventing cancer, the method comprising administering to a mammal in need thereof a therapeutically effective amount of a combination of:
Pioglitazone, or a pharmaceutically acceptable salt thereof; and
a MEK inhibitor, or a pharmaceutically acceptable salt thereof.

In another aspect the present invention provides a kit comprising:
(a) A first pharmaceutical composition comprising Pioglitazone, or a pharmaceutically acceptable salt thereof;
(b) A second pharmaceutical composition comprising a MEK inhibitor or a pharmaceutically acceptable salt thereof; and optionally
(c) Instructions for use in the treatment or prevention of cancer.

In some embodiments, the MEK inhibitor is trametinib, trametinib dimethyl sulfoxide, binimetinib, selumetinib or cobimetinib, or a stereoisomer, mixture of stereoisomers, tautomer, isotopolog, or pharmaceutically acceptable salt thereof.

In one embodiment, said Pioglitazone, is a stereoisomer, mixture of stereoisomers, tautomer, isotopolog, or pharmaceutically acceptable salt thereof.

In one embodiment, the combination comprises or consists of Pioglitazone and cobimetinib.

In one embodiment, Pioglitazone and the MEK inhibitor are administered concurrently.

In one embodiment, the Pioglitazone and the MEK inhibitor are co-formulated.

In one embodiment, the Pioglitazone and the MEK inhibitor are co-formulated in a pharmaceutical composition, further comprising a pharmaceutically acceptable carrier, diluent or excipient.

In one embodiment, the Pioglitazone and the MEK inhibitor are administered sequentially.

In one embodiment, the Pioglitazone and the MEK inhibitor are formulated in separate dosage forms, i.e., as separate pharmaceutical compositions.

In one embodiment, cancer is selected from the group consisting of breast cancer, pancreatic cancer, ovarian cancer and colon cancer.

In one embodiment, breast cancer is triple-negative breast cancer (TNBC).

In one embodiment, said mammal is a human patient.

In another aspect, the present invention provides a combination of a PPARγ agonist and binimetinib, cobimetinib, or selumetinib or a pharmaceutically acceptable salt thereof, for use in treating or preventing cancer, said treating or preventing cancer comprises administering to a mammal in need thereof a therapeutically effective amount of said combination.

In yet another aspect, the present invention provides a method of treating or preventing cancer, the method comprising administering to a mammal in need thereof a therapeutically effective amount of a combination of:
a PPARγ agonist; and
one or more of binimetinib, cobimetinib, selumetinib, or pharmaceutically acceptable salts thereof.

In one embodiment, said PPARγ agonist is Rosiglitazone.

### BRIEF DESCRIPTION OF THE DRAWINGS

To understand the invention and to see how it may be carried out in practice, a preferred embodiment will now be described, by way of non-limiting example only, with reference to the accompanying drawings, in which:

FIG. 1A is a schematic representation describing an optimized protocol to induce MTΔECad cells trans-differentiation into adipocytes with BMP2 and Pioglitazone.

FIG. 1B is a graph showing the percentage of PPARγ positive cells out of MTΔECad cells treated for adipogenesis with BMP2 and increasing concentrations of Pioglitazone for 5 days.

FIG. 2A-2J shows photographs of MTΔECad cells incubated for 10 days with 200 ng/ml BMP2 and either 2 μM Rosiglitazone (Rosi) (FIGS. 2B, 2G) or 10 (FIGS. 2C, 2H), 20 (FIGS. 2D, 2I), or 40 μM (FIGS. 2E, 2J) Pioglitazone (Pio). Control cells were treated with DMSO (FIGS. 2A, 2F). FIGS. 2A-2E are immunofluorescence images of cells stained with anti C/EBPα antibody and Phalloidin that stains actin fibers. FIGS. 2F-2J are brightfield (BF) images of the same field respectively. Bar=100 μm.

FIG. 2K is a graph showing the percentage of C/EBPα positive cells in DMSO (control), BMP2+Rosiglitazone (Rosi) or BMP2+Pioglitazone (Pio) treated cells. Two tailed t-test indicated no statistically significant difference between Rosi and Pio treatments (***, P<0.001; N.S., P>0.05).

FIGS. 2L-2O shows photographs of MTΔECad Cells treated for 10 days with DMSO (Control, FIGS. 2L and 2M) or with BMP2+Pioglitazone (Adipo, FIGS. 2N and 2O). FIGS. 2L and 2N are fluorescence images of cells that were stained with antibodies against Perilipin, BODIPY and DAPI. FIGS. 2M and 2O are brightfield (BF) images of the same cells respectively. Bar=10 μm.

FIGS. 2P-2U are representative images of MTΔECad cells treated for 10 days with DMSO (FIGS. 2P, 2S), BMP2+Rosiglitazone (FIGS. 2Q, 2T) or BMP2+Pioglitazone (FIGS. 2R, 2U). FIGS. 2P, 2Q, 2R show 5-ethynyl-20-deoxyuridine (EdU) staining, that marks proliferating cells. FIGS. 2S, 2T, 2U show overlay with DAPI staining. Bar=100 μm.

FIG. 3A is a schematic description of induction protocol for MTΔECad cancer cells adipogenesis in the presence of TGFβ with BMP2, Pioglitazone and a MEK inhibitor (MEKi).

FIGS. 3B-3O are representative images of MTΔECad cells that were treated as indicated in (FIG. 3A) with BMP2+Pioglitazone (BMP2+Pio) (FIGS. 3C and 3J), BMP2+Pioglitazone and TGFβ (FIGS. 3D and 3K), or BMP2+Pioglitazone+TGFβ together with MEK inhibitor: 0.5 ng/ml Trametinib (FIGS. 3E and 3L), 400 nM Binimctinib (FIGS. 3F and 3M), 0.25 μM Selumetinib (FIGS. 3G and 3N) or 0.25 μM Cobimetinib (FIGS. 3H and 3O) as indicated. Control cells were treated with DMSO (FIGS. 3B and 3I). After 10 days, cells were fixed, stained with BODIPY to mark lipid droplets and with DAPI, and imaged with confocal microscope. FIGS. 3B-3H are fluorescence images. FIGS. 3I-3O are DIC (differential interference contrast) images of the same fields. Bar=20 μm.

FIGS. 4A-4F are representative images of MTΔECad cells that were treated for 10 days according to the adipogenesis protocol described in FIG. 3A. The cells were treated with BMP2, Pioglitazone, TGFβ and different concentrations of Binimetinib, as indicated. After 10 days of treatment, cells were fixed, stained with BODIPY and with DAPI, and imaged with confocal microscope. FIGS. 4A, 4C and 4E are fluorescence images. FIGS. 4B, 4D and 4F are DIC (differential interference contrast) images of the same fields shown in FIGS. 4A, 4C and 4E, respectively. Bar=20 μm.

FIGS. 4G-4L are images of MTΔECad cells that were treated for 10 days according to the adipogenesis protocol described in FIG. 3A. The cells were treated with BMP2, Pioglitazone, TGFβ and different concentrations of Selumetinib, as indicated. After 10 days of treatment, cells were fixed, stained with BODIPY and with DAPI, and imaged with confocal microscope. FIGS. 4G, 4I and 4K are fluorescence images. FIGS. 4H, 4I and 4L are DIC (differential interference contrast) images of the same fields shown in FIGS. 4G, 4I and 4K, respectively. Bar=20 μm.

FIG. 4M-4R are images of MTΔECad cells that were treated for 10 days according to the adipogenesis protocol described in FIG. 3A. The cells were treated with BMP2, Pioglitazone, TGFβ and different concentrations of Cobimetinib, as indicated. After 10 days of treatment, cells were fixed, stained with BODIPY and with DAPI, and imaged with confocal microscope. FIGS. 4M, 4O and 4Q are fluorescence images. FIGS. 4N, 4P and 4R are DIC (differential interference contrast) images of the same fields shown in FIGS. 4M, 4O and 4Q, respectively. Bar=20 μm.

FIGS. 5A-5J are fluorescence images of MTΔECad cells that were treated with BMP2+Pioglitazone (Pio)+TGFβ, together with various MEK inhibitors: tramctinib—FIGS. 5B and 5G, Binimetinib—FIGS. 5C and 5H, Selumetinib—FIGS. 5D and 5I, and Cobimctinib—FIGS. 5E and 5J. FIGS. 5A-5E show 5-ethynyl-20-deoxyuridine (EdU) staining, that marks proliferating cells. FIGS. 5F-5J show overlay with DAPI staining.

FIGS. 6A-6H are representative images of MTΔECad cells. FIGS. 6A-6D are fluorescence images of MTΔECad cells that were treated for 10 days with BMP2+Pioglitazone (BMP2+Pio), BMP2+Pioglitazone and TGFβ, or BMP2+Pioglitazone+TGFβ together with the MEK inhibitor Cobimetinib. Cells were immunostained with antibodies against Perilipin, and counterstained with BODIPY to mark lipid droplets, and with DAPI. FIGS. 6E-6H are brightfield (BF) images of the same fields respectively. Bars=20 μm.

FIGS. 6I-6P are representative images of MTΔECad cells. FIGS. 6I-6L are fluorescence images of MTΔECad cells that were treated for 10 days with BMP2+Rosiglitazone (BMP2+Pio), BMP2+Rosiglitazone and TGFβ, or BMP2+Rosiglitazone+TGFβ together with the MEK inhibitor Cobimetinib. Cells were immuno-stained with antibodies against PPARγ, and counterstained with BODIPY to mark lipid droplets, and with DAPI. FIGS. 6M-6P are brightfield (BF) images of the same fields respectively. Bars=20 μm.

FIGS. 7A-7H are images of tissue sections taken from 3D tissues obtained from core needle biopsy from the tumor of a triple negative breast cancer patient that were treated for 5 days with: DMSO (control) (FIG. 7A); Pioglitazone (Pio) (FIG. 7C), Cobimetinib (FIG. 7E), or a combination of Pioglitazone and Cobimetinib (Pio+Cobimetinib) (FIG. 7G). Tissues were fixed and IHC staining with antibody against PPARγ was performed. The tissue sections were counterstaining with Hematoxylin. Enlargement of the squared areas is shown respectively in FIGS. 7B, 7D, 7F and 7H. Bar=50 μm.

FIG. 7I is a graph showing the percentage of PPARγ positive cells in treated tissues.

FIGS. 8A-J are images of KPC550 cells (a pancreatic tumor cell line). FIGS. 8A-8E are representative images of DMSO-treated cells that served as controls. FIGS. 8F-8J are representative images of KPC550 cells treated for 10 days with BMP2/BMP4 and Rosi/Pioglitazone to induce adipogenesis. FIGS. 8A and 8F are immunofluorescence images of cells stained for the early adipogenesis marker C/EBPα; FIGS. 8B and 8G are immunofluorescence images of cells stained for the early adipogenesis marker PPARγ. The cells were also stained with Phalloidin showing the actin structure. FIGS. 8C and 8H are DIC images revealing intracellular lipid droplets in C/EBPα positive cells; FIGS. 8D and 8I are DIC images revealing intracellular lipid droplets in PPARγ-positive cells. FIGS. 8E and 8J are immunofluorescence images of cells stained for perilipin, a mature adipocyte marker localized to lipid droplet membranes. Nuclei are counterstained with DAPI. Scale bar, 50 μm.

FIGS. 9A-9D are images of patient-derived breast cancer organoids. FIGS. 9A and 9B are representative images of control organoids treated with DMSO. FIGS. 9C and 9D are representative images of organoids treated with Pioglitazone and cobimetinib which trans-differentiated into adipocytes (Adipo). FIGS. 9A and 9C show staining with an antibody to perilipin and DAPI staining to visualize DNA. FIGS. 9B and 9D show DIC staining. Scale bar, 50 μm.

### DETAILED DESCRIPTION OF EXEMPLARY EMBODIMENTS

The present invention is based on the surprising finding that a treatment protocol combining Pioglitazone and a MEK inhibitor effectively induced trans-differentiation and adipogenesis of cancer cells.

As shown in the Examples, murine breast cancer cells and pancreatic cancer cells were treated with Pioglitazone alone and in combination with various MEK inhibitors, including Cobimetinib, Binimetinib, Selumetinib, and Trametinib, at different dose levels to evaluate their effectiveness in inducing adipogenesis. These combinations showed a synergistic effect on adipogenesis. Furthermore, adipogenesis and PPARγ activation was also demonstrated in patient-derived ex-vivo tissue cultures, and organoids.

The tumor microenvironment is characterized by the presence of multiple cytokines, including TGFβ which enhances the cells' plasticity and activates the non-canonical MEK-ERK pathway which inhibits adipogenesis. Without wishing to be bound by theory, the use of MEK inhibitors together with Pioglitazone (which activates PPARγ) overrides this inhibition. Indeed, as shown in the Examples below, administration of MEK inhibitors in combination with Pioglitazone facilitated adipogenesis of cancer cells even in the presence of TGFβ, thus overcoming its inhibitory effect.

The present invention thus provides Pioglitazone and MEK inhibitors, particularly cobimetinib, as a combined treatment of cancer.

The invention is described in the following detailed description with reference to therapeutic methods for the treatment of cancer involving administration of Pioglitazone and the one or more MEK inhibitors to a mammal in need of same.

Thus, in one aspect, the present invention provides a combination of Pioglitazone or a pharmaceutically acceptable salt thereof and a MEK inhibitor or a pharmaceutically acceptable salt thereof, for use in treating or preventing cancer, said treating or preventing cancer comprises administering to a mammal in need thereof a therapeutically effective amount of said combination.

As used herein, the term “combination” refers to concomitant use or administration of Pioglitazone and at least one MEK inhibitor for the treatment of cancer.

The concomitant administration of Pioglitazone and the one or more MEK inhibitors can be simultaneous (namely, given to patients at the same time, i.e., concurrently) or sequential in either order during the entire, or portions of, the treatment period.

The Pioglitazone and the at least one MEK inhibitor may be administered as two separate dosage forms or may be co-formulated in a single dosage form.

Pioglitazone and the MEK inhibitor may be administered following the same or different dosing regimens.

Pioglitazone is known as an anti-diabetic medication used to treat type 2 diabetes and is readily available. Pioglitazone belongs to the thiazolidinedione (TZD) drug class and has the chemical formula C₁₉H₂₀N₂O₃S:

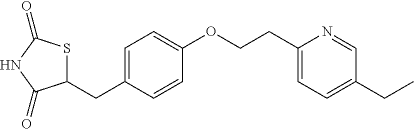 [SMILES: CCc1ccc(CCOc2ccc(CC3SC(=O)NC3=O)cc2)nc1]

MEK inhibitors are known in the art and are readily available. A MEK inhibitor is a compound that inhibits the mitogen-activated protein kinase enzymes MEK1 and/or MEK2. Non-limiting examples of MEK inhibitors include:

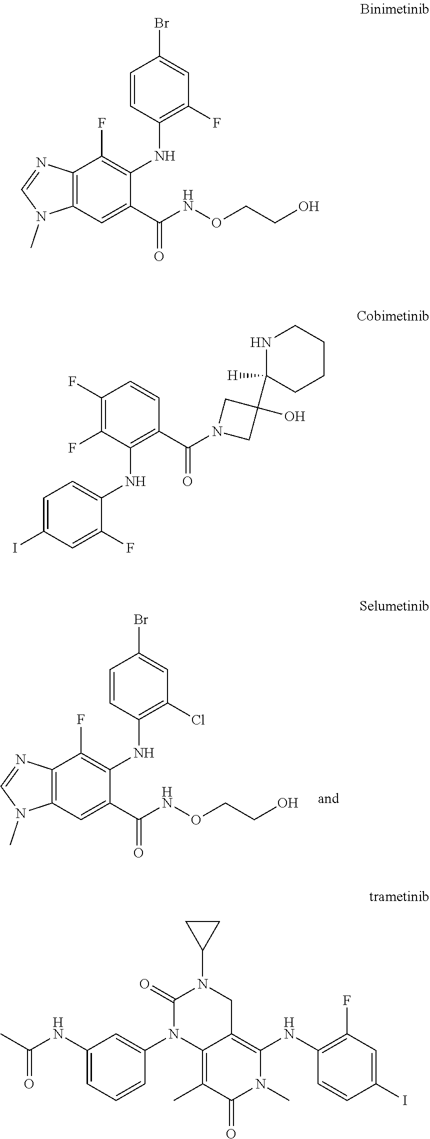 [SMILES: Cn1cnc2c(F)c(Nc3ccc(Br)cc3F)c(C(=O)NOCCO)cc21 ; O=C(c1ccc(F)c(F)c1Nc1ccc(I)cc1F)N1CC(O)([C@@H]2CCCCN2)C1 ; Cn1cnc2c(F)c(Nc3ccc(Br)cc3Cl)c(C(=O)NOCCO)cc21 ; CC(=O)Nc1cccc(N2C(=O)N(C3CC3)Cc3c2c(C)c(=O)n(C)c3Nc2ccc(I)cc2F)c1]

The present disclosure also encompasses pharmaceutically acceptable salts of Pioglitazone or a MEK inhibitor. A “pharmaceutically acceptable salt” refers to any non-toxic alkali metal, alkaline earth metal, and ammonium salt commonly used in the pharmaceutical industry, including the sodium, potassium, lithium, calcium, magnesium, barium ammonium and protamine zinc salts, which are prepared by methods known in the art. The term also includes non-toxic acid addition salts, which are generally prepared by reacting the ligand with a suitable organic or inorganic acid. The acid addition salts are those which retain the biological effectiveness and qualitative properties of the free bases, and which are not toxic or otherwise undesirable. Examples include, inter alia, acids derived from mineral acids, hydrochloric, hydrobromic, sulfuric, nitric, phosphoric, metaphosphoric and the like. Organic acids include, inter alia, tartaric, acetic, propionic, citric, malic, malonic, lactic, fumaric, benzoic, cinnamic, mandelic, glycolic, gluconic, pyruvic, succinic salicylic and arylsulphonic, e.g., p-toluenesulphonic, acids.

The disclosure further encompasses a stereoisomer, mixture of stereoisomers, tautomer, or isotopolog, of Pioglitazone and of the MEK inhibitor.

In another embodiment, the present invention also provides a combination of a PPARγ agonist and binimetinib, cobimetinib, or selumetinib as a combined treatment of cancer.

In another embodiment, the present invention provides a combination of Rosiglitazone and binimetinib, cobimetinib, or selumetinib as a combined treatment of cancer.

The term “mammal” includes, but is not limited to, humans, domestic and farm animals, and zoo, sports or pet animals such as guinea pigs, monkeys, dogs, cats, horses, cows, pigs, and sheep.

Herein, a “patient” (interchangeably termed “individual” or “subject”) is a human male or a human female. In an embodiment, the patient is a “cancer patient”, i.e. one who is diagnosed with cancer and is or will be receiving, or has received, medical care for treating the cancer. In another embodiment, the patient is a subject in a high risk for cancer, for example, but not limited to carriers of the BRCA mutation.

The term “cancer” refers to or describes the physiological condition in mammals that is typically characterized by unregulated cell growth. The therapeutic methods and compositions of the invention may be applicable to the treatment or prevention of various types of cancer, including but not limited to breast cancer, pancreatic cancer, ovarian cancer or colon cancer, and including aggressive cancers such as triple-negative breast cancer (TNBC).

The Pioglitazone and the MEK inhibitor are administered in amounts which are sufficient to achieve a desired therapeutic effect. The desired therapeutic effect may include, without being limited thereto, reduction in tumor or metastasis volume, induction of adipogenesis, prevention of tumor formation or metastasis, and/or an increase in the patient's survival rate after surgical resection of the tumor.

As will be appreciated, the amount of the Pioglitazone and the MEK inhibitor will depend on the severity of the disease, the intended therapeutic regimen, and the desired therapeutic dose.

An amount effective to achieve the desired effect is determined by considerations known in the art. An “effective amount” for purposes herein must be effective to achieve a therapeutic effect, the therapeutic effect being as defined hereinbefore.

It is appreciated that the effective amount depends on a variety of factors including a variety of pharmacological parameters such as half-life in the body, undesired side effects, if any, factors such as age and gender of the subject to be treated, etc. The effective amount is typically tested in clinical studies having the aim of finding the effective dose range, the maximal tolerated dose, and the optimal dose. The manner of conducting such clinical studies is well known to a person versed in the art of clinical development.

An amount may also at times be determined based on amounts shown to be effective in animals. It is well known that an amount administered to animals (e.g., mice) can be converted to an equivalent amount in another species (notably humans) using one of possible well-known conversion equations.

In some embodiments Pioglitazone and the one or more MEK inhibitors are formulated or co-formulated in a pharmaceutical composition, further comprising a pharmaceutically acceptable carrier or excipient.

Thus, in one embodiment the present invention provides a pharmaceutical composition comprising Pioglitazone and a MEK inhibitor and a pharmaceutically acceptable carrier, diluent or excipient. In an embodiment, the pharmaceutical composition is for use in the treatment of cancer in a mammalian subject, e.g., a human patient.

In other embodiments, the present invention provides a pharmaceutical composition comprising Pioglitazone and a pharmaceutically acceptable carrier or diluent, for use in combination with a MEK inhibitor.

In other embodiments, the present invention provides a pharmaceutical composition comprising a MEK inhibitor and a pharmaceutically acceptable carrier or diluent, for use in combination with Pioglitazone.

A “pharmaceutical composition” in the context of the invention is intended to mean a combination of the active agent(s) (i.e., Pioglitazone and one or more MEK inhibitors), together or separately, with a pharmaceutically acceptable carrier as well as other additives. The carrier may at times have the effect of improving the delivery or penetration of the active ingredient(s) to the target tissue, improving the stability of the drug(s), slowing clearance rates, imparting slow-release properties, reducing undesired side effects etc. The carrier may also be a substance that stabilizes the formulation (e.g., a preservative). Examples of carriers, stabilizers, and adjuvants, can be found in E. W. Martin, REMINGTON'S PHARMACEUTICAL SCIENCES, MacK Pub Co (June 1990).

The term “pharmaceutically acceptable carrier” in the context of the present invention denotes any one of inert, non-toxic materials, which do not react with the Pioglitazone and the MEK inhibitor, and which can be added to formulations as diluents, carriers or to give form or consistency to the formulation.

In another aspect, the present invention provides a method of treating or preventing cancer, the method comprising administering to a patient in need thereof a therapeutically effective amount of a combination of:
Pioglitazone, or a pharmaceutically acceptable salt thereof; and
a MEK inhibitor, or a pharmaceutically acceptable salt thereof.

The term “a method of treating cancer” or its equivalent refers to a procedure or course of action that is designed to reduce or eliminate the number of cancer cells in a patient, or to alleviate the symptoms of cancer.

In the context of the present invention the term “treating cancer”, or “treatment of cancer” comprises treating a cancer patient to reverse, attenuate, alleviate, inhibit the progress of, or prevent, either partially or completely, the growth of tumors, tumor metastases, or other cancer-causing or neoplastic cells in a patient.

The term “preventing cancer” encompasses administering the combination of the present invention as a prophylactic measure to subjects that are not yet afflicted with the disease but are in a high risk for cancer, for example, but not limited to, carriers of the BRCA mutation. The term also encompasses administering the combination of the present invention as a prophylactic measure to subjects that recovered from cancer and are at risk for disease recurrence.

Pioglitazone and the MEK inhibitor of the present invention can be administered in a single dose (namely, a one-time medication) or as a continuous treatment, for a period of days, weeks, months or even years.

The Pioglitazone and the MEK inhibitor of the present invention are administered and dosed in accordance with good medical practice, taking into consideration the clinical condition of the individual patient, the site and method of administration, scheduling of administration, patient age, sex, body weight and other factors known to medical practitioners. The choice of carrier will be determined in part by the specific active ingredient, as well as by the specific method used to administer the composition. Accordingly, there is a wide variety of suitable pharmaceutical compositions of the present invention.

As used herein, the terms “administer”, “administering”, “administered”, or “administration” refer to providing a compound or a composition (e.g., the combination described herein), to a subject or patient. The Pioglitazone and the MEK inhibitor of the invention can be administered to the patient by a variety of delivery modes as known in the art, e.g., by oral, intraperitoneal, subcutaneous, transcutaneous, topical, intramuscular, intraarticular, subconjunctival, intranasal, or intraocular administration. The carrier will be selected based on the desired form of the formulation.

In an embodiment, Pioglitazone and the MEK inhibitor are provided to the patient by oral administration, using any means suitable for oral administration, for example by a tablet, e.g., a film-coated tablet.

In an embodiment, the administration is by an extended-release formulation.

The therapeutic use of Pioglitazone and the one or more MEK inhibitors may at times be in combination with additional drugs or therapeutic procedures such as removal of the tumor (or in the case of breast cancer, complete breastectomy), irradiation, immunotherapy, therapy with biological drugs, and/or chemotherapy. Immunotherapy may include the administration of an anti-tumor antibody and/or a checkpoint inhibitor. Chemotherapy may include, but is not limited to, nucleoside chemotherapeutics (e.g., gemcitabine), fluoropyrimidine (fluorouracil plus leucovorin), or fluorouracil, plus leucovorin plus irinotecan plus oxaliplatin (known as FOLFIRINOX). The additional drugs may include other adipogenesis inducing agents such as Rosiglitazone, Insulin, or Dexamethasone.

The additional drug may be given to patients at the same time as the Pioglitazone and MEK inhibitors combination or at different times, before or after administration of the additional drug, depending on the dosing schedule of each of the drugs.

As used in the specification and claims, the forms “a”, “an” and “the” include singular as well as plural references unless the context clearly dictates otherwise. For example, the term “a MEK inhibitor” includes one or more MEK inhibitors.

Further, as used herein, the term “comprising” is intended to mean that the method or composition includes the recited elements but does not exclude others. Similarly, “consisting essentially of” is used to define methods and compositions that include the recited elements but exclude other elements that may have an essential significant therapeutic activity towards cancer. “Consisting of” shall mean excluding more than trace elements of other elements. Embodiments defined by each of these transition terms are within the scope of this invention.

In another aspect the present invention provides Pioglitazone or pharmaceutically acceptable salt thereof for use in a method of treating cancer, wherein the method comprises administering to a mammal in need thereof a therapeutically effective amount of said Pioglitazone or pharmaceutically acceptable salt thereof in combination with a MEK inhibitor, or pharmaceutically acceptable salt thereof.

### EXEMPLARY EMBODIMENTS

### Materials & Methods

### Cell Culture

MTΔECad cells (A. Fantozzi et al., (2014) Cancer research, vol. 74, no. 5, pp. 1566-75) were cultured in high glucose Dulbecco's modified Eagle Medium (DMEM (Sartorius; 01-055-1A)) supplemented with 10% Fetal Bovine Serum (FBS; A5256701; ThermoFisher), ×1 Penicillin/streptomycin (L0022-100; Biowest) and 2 mM L-Glutamine (X0550-100; Biowest). Cells were grown at 37° C., 5% CO₂, 95% humidity. KPC cells were maintained in DMEM (high glucose) (01-052-1A; Sartorius, Göttingen, Germany) supplemented with 10% Fetal Bovine Serum (FBS; A5256701; ThermoFisher, Waltham, MA, USA), ×1 Penicillin/streptomycin (L0022-100; Biowest, Nuaillé, France), and Sodium Pyruvate (L0642-100; Biowest, Nuaillé, France).

### Cancer Adipogenesis

For cancer adipogenesis experiments, MTΔECad cells were seeded at density of 20,000 cells/cm² in 18-well μ Slide (81816; ibidi) and incubated overnight at 37° C. in 5% CO₂. Then, cells were treated with 200 ng/ml human recombinant BMP2 (B3555, Sigma) for 3 days, with 200 ng/ml BMP2 and 2 μM Rosiglitazone (AG-CR1-3570-M010; Adipogen) or Pioglitazone (71745-50; Cayman Chemical) for 4 days, and with medium containing 2 μM Rosiglitazone or Pioglitazone as indicated for additional 3 days.

For MEK inhibitor experiments, MTΔECad cells were seeded in ibidi 18-well μ Slide at density of 30,000 cells/cm² and incubated overnight. Then, cells were treated for 3 days with 200 ng/ml human recombinant BMP2, 2 ng/ml TGFβ (R&D Systems, 240-B), and MEK inhibitors (Trametinib; BNT-8123, Selumetinib; BNS-4490, Binimetinib; B-2332, Cobimetinib; BNC-1100; Lc Laboratories) as indicated. Then, cells were treated for 4 days with BMP2, TGFβ, MEK inhibitors and 20 μM Pioglitazone. Finally, cells were treated for 3 days with Pioglitazone and fixed with 4% Paraformaldehyde (PFA). Control cells were treated with medium containing DMSO.

For trans differentiation of KPC500 cells, cells were plated in a density of 100 cell3/0.34 cm². cells were first treated with BMP2/BMP4 for 3 days, followed by BMP2/BMP4 plus Rosi/Pioglitazone for 4 days, and then continued with Rosi/Pioglitazone alone for 3 additional days.

For trans-differentiation of patient-derived breast cancer organoids into adipocytes, triple negative breast cancer organoids were plated in 18-well μ Slide (81816; ibidi) and treated for 3 weeks with 10 μM pioglitazone and 0.25 μM cobimetinib, control organoids were treated with DMSO.

### Cell Proliferation Assay (EdU)

MTΔECad cells were treated for 10 days with 200 ng/ml BMP2 and 2 μM Rosiglitazone (Rosi) or 10 μM Pioglitazone (Pio), with or without 2 ng/ml TGFβ and MEK inhibitors (0.5 ng/ml Trametinib, 400 μM Binimetinib, 0.25 μM Selumetinib or 0.25 μM Cobimetinib). On day 7 of adipogenesis protocol, cells were incubated for 72 hours with 5 μM Click-iT EdU (C10337, ThermoFisher). ClickIT reaction was performed according to the manufacturer's protocol, and nuclear staining was performed with 4′,6-diamidino-2-phenylindole (DAPI; D1306; Invitrogen) for 10 minutes. Cells were washed with PBS, mounted with ibidi mounting medium (IBD-50001; ibidi) and imaged with fluorescent microscope.

### Ex-Vivo Cultures of Tumor Tissues

Tumor tissue was obtained from a metastatic lymph node core needle biopsy of a 37 years old metastatic triple negative breast cancer patient upon informed consent. The tissue was cut into 250 μm slices using a vibratome (VF300, Precisionary Instruments).

A sample of the tissue was fixed immediately in 4% paraformaldehyde (PFA) as a reference and analyzed for viability within 24 h. The rest of the slices were placed in 12 or 24 well plates on titanium grids with 4 ml of DMEM/F12 medium (D6421, Sigma) [supplemented with 5% fetal calf serum (FCS) (10270-106, Gibco), penicillin 100 IU/ml with streptomycin 100 μg/ml (15140-122, ThermoFisher), Amphotericin B (A2942, Sigma) 2.5 μg/ml, 50 mg/ml Gentamicin sulfate (G1397, Sigma), and L-glutamine 100 μL/ml (03-020-1B, Sartorius)]. The tissue slices were then cultured at 70 rpm on an orbital shaker (TOU-120) at 37° C., 5% CO₂, and 80% O₂. One day after sectioning, tumor sections were treated with drugs and were cultured for 96 h with medium change after 48 h. At the end of the incubation period the tissue sections were fixed overnight with 4% PFA followed by formalin-fixed paraffin embedding (FFPE). Drug concentrations: Cobimetinb 1.25 μM-2.5 μM; Pioglizonate (20 μM-100 μM).

### Immunohistochemistry (IHC)

IHC staining was performed on 4 μm FFPE sections using the Leica Bond max system (Leica Biosystems Newcastle Ltd, UK). Slides were baked for 30 min at 60° C., dewaxed and pretreated for 20 minutes with epitope-retrieval solution (ER2, Leica Biosystems Newcastle Ltd, UK) followed by incubation with anti PPARγ antibody (Santa Crus SC-7273, 1:50). Detection was performed using the Leica Bond Polymer Refine HRP kit (Leica Biosystems Newcastle Ltd, UK). All slides were counter-stained with Hematoxylin.

### Immunofluorescence

Cells were plated in an 18-well μ Slide (81816; ibidi) and covered with appropriate growth medium overnight. Following treatments, cells were fixed in 4% paraformaldehyde for 20 minutes, permeabilized with 0.5% Triton X100 (93443, Sigma Aldrich) in phosphate buffered saline (PBS; 02-023-1A; Sartorius) for 2 min, and blocked with 5% Bovine serum albumin (BSA; Avantor; 0332-50G) in PBS for 20 min. The cells were incubated with primary antibody diluted 1:100 in PBS (Rabbit anti PPARγ, CST-2443S; Rabbit anti C/EBPα, CST-8178S; or rabbit anti Perilipin, CST-9349S; Cell Signaling) for 1 hour at room temperature (RT), and washed three times for 5 min with PBS at RT. Then cells were incubated with secondary antibody diluted 1:500 in PBS (Alexa Fluor 647 labeled goat anti Rabbit (ab150083; abcam); Alexa Fluor 488 labeled Goat Anti-Rabbit (ab150077; abcam) or Alexa Fluor 555 labeled Donkey Anti-Rabbit (ab150062; abcam)) for 1 hour at RT, together with Phalloidin-iFluor 647 Reagent (ab176759; abcam) (1:1000) or BODIPY FL C12 (D3822; Thermo Fisher) (1:5000) as indicated in figure legends. Cells were washed three times for 5 min with PBS at RT, and stained with 4′,6-diamidino-2-phenylindole (DAPI; D1306; Invitrogen) for 10 min to mark cell nuclei. Finally, cells were rapidly washed with PBS and covered with ibidi mounting medium (IBD-50001; ibidi) or PBS.

### Microscopy

Confocal and widefield fluorescent imaging was performed on TCS SP8 Leica Confocal microscope, equipped with Leica DFC9000 GT camera (Leica Microsystems, Germany).

IHC slides were scanned at ×40 magnification using VENTANA® DP 200 slide scanner (Roche Diagnostics).

### Image Processing and Analysis

Image processing (i.e. Crope, pseudo-coloring, merge channels) was performed by LAS X software and by the Fiji processing software package of ImageJ. For quantification of PPARγ and C/EBPα positive cells, the DAPI and PPARγ/C/EBPα channels of individual fields were thresholded by ImageJ, and single nuclei were counted using the ‘Analyze Particles’ feature. The percentage of PPARγ/C/EBPα count out of the DAPI count was calculated for every field, and the average percentages for every condition were plotted using Microsoft Excel software. Analysis of PPARγ positive cells in IHC images was done using QuPath 5.0.

### Statistical Analysis

Statistical comparisons between groups were performed using a two-tailed t test in Microsoft Excel software.

### Example 1: Pioglitazone Induces Cancer Cells Trans-Differentiation into Adipocytes

Epithelial MTflECad cells (with floxed E-cadherin) are derived from a murine MMTV-neu driven primary tumor. Upon Cre recombination the cells undergo E-cadherin ablation and become irreversibly mesenchymal, generating the MTΔECad cells (D. Ishay-Ronen et al., (2019) Cancer cell, vol. 35, no. 1, pp. 17-32; A. Fantozzi et al., (2014) Cancer research, vol. 74, no. 5, pp. 1566-75)]. MTΔECad cells can undergo adipogenesis by activating MET with BMP2 (BMP2 is required only in in vitro protocol D. Ishay-Ronen et al., (2019)) and Rosiglitazone. When TGFβ is added to the protocol, a combination with a MEK inhibitor is required to induce adipogenesis (D. Ishay-Ronen et al., (2019)). Thus, these cells serve as an efficient model to study isolated drug effects.

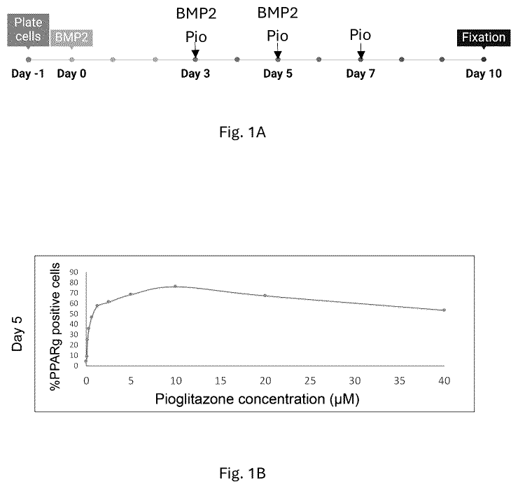

Murine MTΔECad breast cancer cells were induced to undergo trans-differentiation into adipocytes according to a protocol combining BMP2 with increasing concentrations of Pioglitazone. The adipogenesis induction protocol is shown in FIG. 1A. MTΔECad cells were treated to induce adipogenesis for 5 days with BMP2 and 5, 10, or 20 μM Pioglitazone (Pio). Negative control cells were treated with DMSO. Cells were then fixed and immunostained with antibodies against the two major adipogenesis-related transcription factors PPARγ and C/EBPα and imaged with confocal microscope. Pioglitazone upregulated both transcription factors in a dose dependent manner, as demonstrated by immunolabeling and confocal microscopy visualization. The effect of Pioglitazone on PPARγ upregulation was measured after 5 days of adipogenesis induction and revealed an ideal dose range from 5 to 20 μM (FIG. 1B).

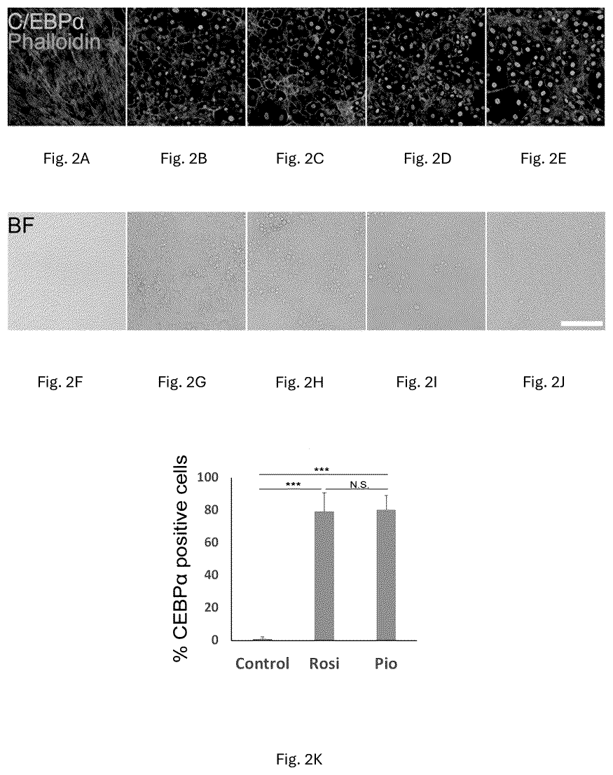

Next, it was shown that in addition to the efficient adipogenesis transcription factors upregulation induced by Pioglitazone in the murine breast cancer cells, Pioglitazone also efficiently induced trans-differentiation of the breast cancer cells into well-differentiated adipocytes. The upregulated C/EBPα correlated with a phenotypic remodeling of the cytoskeleton shifting from mesenchymal stress fibers into cortical actin reorganization (FIGS. 2A-2K). Adipogenesis was induced in MTΔECad cells for 10 days with 200 ng/ml BMP2 and either 2 μM Rosiglitazone (Rosi) or 10, 20, or 40 μM Pioglitazone (Pio) as indicated in FIG. 2A-2J. Control cells were treated with DMSO. Cells were immune-stained with anti C/EBPα antibody and counterstained with Phalloidin to label F-actin, visualizing actin rearrangement from stress fibers into cortical structures. Brightfield (BF) images demonstrate the formation of lipid droplets in the cytoplasm of treated cells. As shown in FIG. 2K C/EBPα positive cells were quantified showing a remarkable increase in the number of positive cells compared with the control. As can be seen in FIGS. 2L-2O, MTΔECad Cells treated for 10 days with DMSO or with BMP2+Pioglitazone were immune stained with antibody against the specific adipocyte marker Perilipin (a lipid droplet membrane protein), and counterstained with BODIPY to visualize lipid droplets. On day 7 of adipogenesis treatment, cells were incubated with 5-ethynyl-20-deoxyuridine (EdU) for 72 hours to label proliferating cells. During cancer cells trans-differentiation, cancer cells underwent growth arrest typical for mature adipocytes (FIGS. 2P-2U).

### Example 2: MEK Inhibitors Facilitate Cancer Trans-Differentiation in the Presence of TGFβ

TGFβ is a potent inducer of epithelial to mesenchymal transition (EMT) in vitro and in vivo. It is also known to impair the development of adipose tissue and inhibit adipogenesis. TGFβ-mediated inhibition of non-cancer adipogenesis is primarily linked to its canonical signaling pathway through SMAD proteins, particularly SMAD3 (Rosen and MacDougald, 2006). Moreover, TGFβ activation of the non-canonical MEK-ERK pathway further interferes with adipogenesis by preventing the differentiation of preadipocytes into mature adipocytes (Isahy-Ronen et al., 2019; Banks et al., 2015)

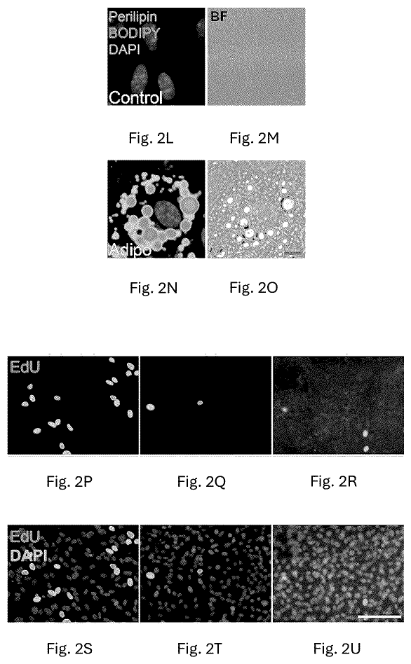

Several MEK inhibitors were administered in combination with Pioglitazone to test their ability to overcome the inhibitory effects of TGFβ and facilitate adipogenesis of cancer cells in the presence of TGFβ. As shown in FIGS. 3A-3O, MEK inhibitors enabled cancer trans-differentiation even in the presence of TGFβ.

FIG. 3A depicts the experimental protocol.
a. MTΔECad cells were grown in the presence of BMP2, TGFβ and a MEK inhibitor, on day 3 and on day 5 the medium was replaced, and the cells were further cultured with BMP2, TGFβ, a MEK inhibitor and Pioglitazone, on day 7 the medium was removed and only Pioglitazone was added. The cells were fixed on day 10.

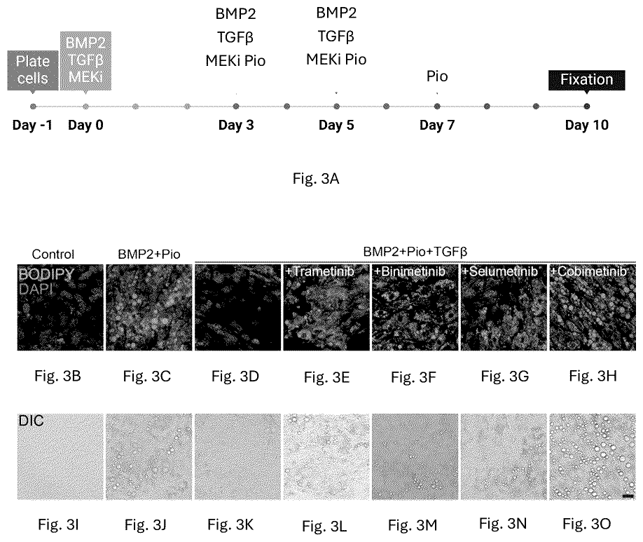

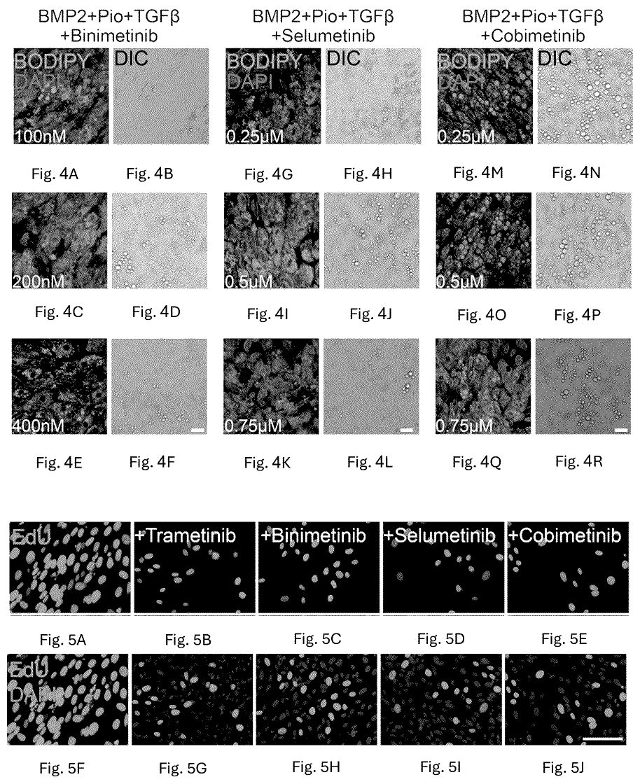

As shown in FIGS. 3B-3O several FDA-approved MEK inhibitors were assessed for their effect on adipogenesis induction including Trametinib (0.5 ng/ml), Binimetinib (400 nM), Selumetinib (0.25 μM) and Cobimetinib (0.25 μM). Adipogenesis induction was also assessed in the presence of increasing doses of the MEK inhibitors (FIGS. 4A-4R). As shown in FIGS. 5A-5J, MEK inhibitors decrease cell proliferation in the TGFβ-treated murine breast cancer cells during trans differentiation. While all tested MEK inhibitors were able to induce adipogenesis, Cobimetinib had a prominent effect on lipid droplets accumulation and proliferation inhibition.

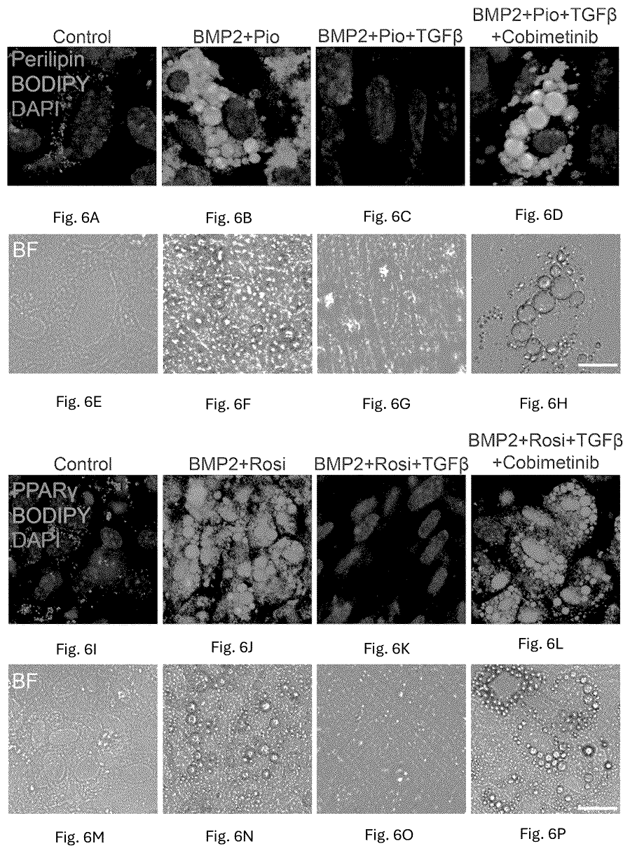

Cobimetinib induced PPARγ upregulation and lipid droplet accumulation with Perilipin expression when combined with Pioglitazone (FIG. 6).

### Example 3: A Synergistic Effect of Pioglitazone and Cobimetinib in PPARγ Upregulation in Patient-Derived Ex-Vivo Tumor Culture

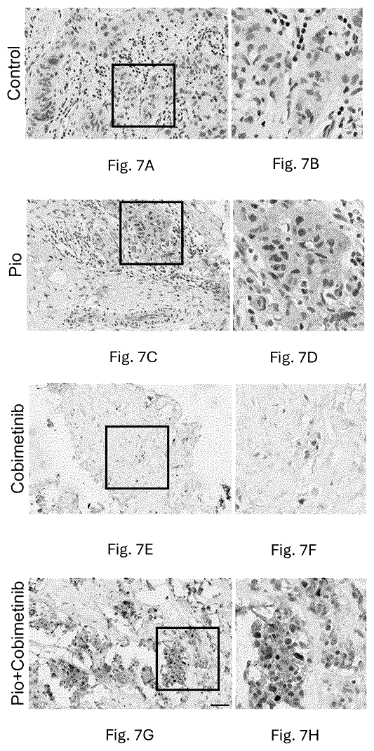

A specialized ex vivo organ culture (EVOC) model, the cResponse platform, was shown to predict drug response in breast cancer patients (Gavert et al., 2022). EVOC can preserve viable cancer tissues with the tumor's intact microenvironment for 5-7 days. To test the possibility to induce adipogenesis in heavily pretreated, metastatic triple negative breast cancer patient, PPARγ expression was evaluated using IHC staining, following 5 days of ex vivo treatment. Pathological evaluation confirmed negative PPARγ staining in control sections and weak IHC staining in Pioglitazone as well as in Cobimetinib treated tissue sections. However, strong PPARγ expression was detected in the metastatic tumor section treated with a combination of Pioglitazone and Cobimetinib. Analysis of percentage of cells positively stained for PPARγ indicated >20 times increase in percentage of positive cells (FIGS. 7A-7I). These results indicate a possible response of heavily pretreated metastatic TNBC to trans-differentiation treatment.

### Example 4: Trans-Differentiation of Mesenchymal Pancreatic Cancer Cells into Adipocytes

KPC550 cell line was established from pancreatic tumors in KPC mice, a well-characterized genetically engineered model of pancreatic ductal adenocarcinoma (PDAC). These mice develop highly penetrant, metastatic pancreatic cancer through pancreas-specific activation of mutant KrasG12D and Trp53R172H alleles, driven by Pdx1-Cre recombinase (Krebs et al., 2017).

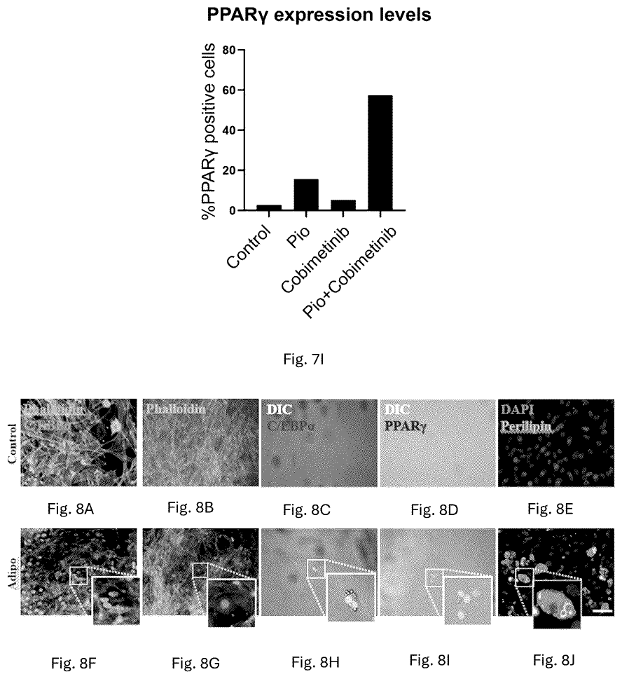

For trans differentiation experiments, KPC550 cells were plated in a density of 100 cells/0.34 cm². The cells were first treated with BMP2/BMP4 for 3 days, followed by BMP2/BMP4 plus Rosi/Pioglitazone for 4 days, and then continued with Rosi/Pioglitazone alone for 3 additional days. DMSO-treated cells served as controls. Cells were fixed with 4% PFA and subjected to immunostaining. As can be seen in FIGS. 8A-8J upregulation of both C/EBPα and PPARγ adipogenesis transcription factors was observed, along with a shift in actin organization from stress fibers to a more cortical pattern in positive cells-consistent with a transition toward a more differentiated state. Notably, intracellular lipid droplets were visible in the cytoplasm of C/EBPα- and PPARγ-positive cells in DIC (Differential Interference Contrast) images. To confirm functional adipocyte differentiation, perilipin expression, a marker of mature adipocytes that localizes to lipid droplet membranes, was assessed and its presence was observed in differentiated cells.

### Example 5: Trans-Differentiation of Patient-Derived Breast Cancer Organoids into Adipocytes

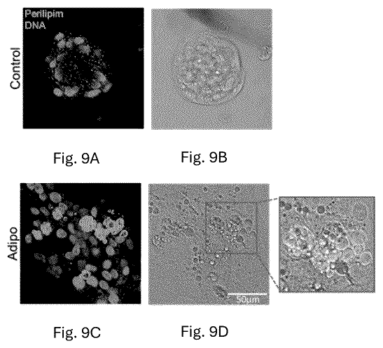

Triple negative breast cancer organoids were treated for 3 weeks with 10 μM Pioglitazone and 0.25 μM cobimetinib, control organoids were treated with DMSO. As can be seen in FIGS. 9A-D, DIC imaging reveals lipid droplets in the cytoplasm of the cells in the treated organoids. Immunostaining with perilipin antibody, a mature adipocyte marker localized to lipid droplet membranes, confirms terminal adipocyte differentiation.

1. A method of treating or preventing cancer, the method comprising administering to a mammal in need thereof a therapeutically effective amount of a combination of:
Pioglitazone, or a pharmaceutically acceptable salt thereof; and
a MEK inhibitor, or a pharmaceutically acceptable salt thereof.

2. The method of claim 1, wherein the MEK inhibitor is trametinib, trametinib dimethyl sulfoxide, binimetinib, selumetinib or cobimetinib, or a stereoisomer, mixture of stereoisomers, tautomer, isotopolog, or pharmaceutically acceptable salt thereof.

3. The method of claim 1 wherein said Pioglitazone, is a stereoisomer, mixture of stereoisomers, tautomer, isotopolog, or pharmaceutically acceptable salt thereof.

4. The method of claim 1 wherein the combination comprises or consisting of Pioglitazone and cobimetinib.

5. The method of claim 1 wherein Pioglitazone and the MEK inhibitor are administered concurrently.

6. The method of claim 1 wherein Pioglitazone and the MEK inhibitor are administered sequentially.

7. The method of claim 1 wherein the cancer is selected from the group consisting of breast cancer, pancreatic cancer, ovarian cancer and colon cancer.

8. The method of claim 7 wherein the breast cancer is triple-negative breast cancer (TNBC).

9. The method of claim 1 wherein said mammal is a human patient.

10. A kit comprising:
(a) A first pharmaceutical composition comprising Pioglitazone, or a pharmaceutically acceptable salt thereof;
(b) A second pharmaceutical composition comprising a MEK inhibitor or a pharmaceutically acceptable salt thereof; and optionally
(c) Instructions for use in the treatment or prevention of cancer.

11. The kit of claim 10, wherein the MEK inhibitor is trametinib, trametinib dimethyl sulfoxide, binimetinib, selumetinib or cobimetinib, or a stereoisomer, mixture of stereoisomers, tautomer, isotopolog, or pharmaceutically acceptable salt thereof.

12. The kit of claim 10 wherein said Pioglitazone, is a stereoisomer, mixture of stereoisomers, tautomer, isotopolog, or pharmaceutically acceptable salt thereof.

13. The kit of claim 10 wherein the kit comprises or consisting of Pioglitazone and cobimetinib.

14. The kit of claim 10 wherein each of the first unit dose and the second unit dose comprises a pharmaceutically acceptable excipient or carrier.

15. The kit of claim 10 wherein the cancer is selected from the group consisting of breast cancer, pancreatic cancer, ovarian cancer and colon cancer.

16. The kit of claim 15 wherein the breast cancer is triple-negative breast cancer (TNBC).

17. A pharmaceutical composition comprising or consisting of Pioglitazone, or a pharmaceutically acceptable salt thereof, a MEK inhibitor or a pharmaceutically acceptable salt thereof, and a pharmaceutically acceptable carrier, diluent or excipient.

18. The pharmaceutical composition of claim 17 wherein said MEK inhibitor is trametinib, trametinib dimethyl sulfoxide, binimetinib, selumetinib or cobimetinib.

19. A method of treating or preventing cancer, the method comprising administering to a mammal in need thereof a therapeutically effective amount of a combination of:
a PPARγ agonist; and
one or more of binimetinib, cobimetinib, selumetinib, or pharmaceutically acceptable salts thereof.

20. The method of claim 19 wherein said PPARγ agonist is Rosiglitazone.
